(19) United States
(12) Patent Application Publication
(10) Pub. No.: US 2025/0152952 A1
(43) Pub. Date: May 15, 2025

(54) AUTOMATED EXTERNAL DEFIBRILLATORS WITH AUTOMATIC SELECTION BETWEEN ADULT AND PAEDIATRIC DEFIBRILLATION DOSES

(71) Applicant: CELLAED LIFE SAVER PTY LTD, Sydney, New South Wales (AU)

(72) Inventors: Donovan Lachlan CASEY, Sydney, New South Wales (AU); Erol Erdogan TEBER, West Ryde Sydney, New South Wales (AU); Atheer NASSIR, Riverwood Sydney, New South Wales (AU)

(21) Appl. No.: 18/836,024

(22) PCT Filed: Feb. 9, 2023
(86) PCT No.: PCT/IB2023/051187

§ 371 Date: Aug. 6, 2024

(30) Foreign Application Priority Data
Feb. 9, 2022 (AU) 2022900264

(51) Int. Cl. A61N 1/39; A61N 1/04; A61N 1/365

(52) U.S. Cl. CPC A61N 1/3931; A61N 1/046; A61N 1/36521; A61N 1/3904; A61N 1/3975

(57) ABSTRACT

Described is an AED and method of use, comprising two pads to be placed on a patient, two electrodes on the two pads respectively, two angular motion sensors associated with the two pads respectively, a shock generation circuit to generate doses of defibrillation shocks, and a processor with memory and power supply, the processor configured to analyse angular motion signals from the two angular motion sensors to determine orientations of the two pads relative to one another, analyse electrocardiogram (ECG) signals from the two electrodes to determine shockable cardiac rhythms, automatically select between adult doses and paediatric doses of defibrillation shocks to be delivered by the two electrodes based, at least in part, on the orientations of the two pads.

### FIELD

The present invention relates generally to automated external defibrillators (AEDs) with automatic selection between adult and paediatric defibrillation doses.

### BACKGROUND

Existing AEDs are configured to provide adult and paediatric doses of defibrillation shocks having different energies using two electrodes on two defibrillation pads respectively. Most AEDs have visual guides for pad placement that rely on placing the electrodes at different specific locations for adult and paediatric patients.

Adult doses of defibrillation shocks are typically delivered with the two pads placed on an adult patient in an anterior-anterior orientation so that one of the two electrodes is a sternal electrode and the other is an apex electrode. Lower-energy paediatric doses of defibrillation shocks are typically delivered with the two pads placed on a child patient in an anterior-posterior orientation so that one of the two electrodes is a front chest electrode and the other is a back electrode.

Conventional AEDs suffer several shortcomings. The selection between adult and paediatric doses for child patients is not fully automated but instead requires caregivers to manually select child doses, and prevent incorrect operation with adult doses. For example, the use of press buttons or key inserts that manually set operation of the AED to “child mode,” or child-specific pads and/or leads that manually hardwire the AED to operate in “child mode.”

One of the key requirements for AEDs is a compact device form factor with small pad footprints. This creates an internal packaging space constraint that limits battery size, which in turn limits the cycle life and shelf life of conventional AEDs.

The arrangements used in conventional AEDs to manually select between adult and paediatric defibrillation doses reduce the internal space available for packaging batteries, which in turn further limits the cycle life and shelf life of compact AEDs.

One such configuration of defibrillator using motion sensors for electrode placement determination and dosage administration is disclosed in EP 3679353.

However, some of the drawbacks of the method and apparatus of EP 3679353 include the following.
The use of separate pads for infant and adult dosages.
EP 3679353 does not use TTI to differentiate between adult and infant/paediatric (as separate pads are used).
The determination of the relative orientation of the pads and spacing limits for pad placements are highly prone to errors due to variations in patient size, chest topography and other factors such as the presence of implantable defibrillators and pacemakers that necessitate moving electrodes out of standard positions.
Anthropometric models used to determine patient size vary significantly by population type and gender, and therefore may not provide an accurate assessment of the patient.
The device of EP 3679353 extends the time taken to administer a shock to the patient, as a result of the requirement of interaction from the user (i.e., confirmation of steps taken or additional steps taken to confirm pad positions).

In view of this background, there is an unmet need for AEDs having improved arrangements for selecting between adult doses and paediatric doses of defibrillation shocks. In addition, there is also an unmet need for AEDs capable of therapeutic use in non-hospital or medical settings, where the users do not have training or expert knowledge on the use of AEDs.

It is an object of the disclosure to provide an improved automatic dosage adjustment system and automated external defibrillator having the same which addresses or ameliorates one or more disadvantages or limitations associated with the prior art, or at least which provides the public with a useful choice.

### SUMMARY

In one aspect the present disclosure may provide an AED with a small form factor having a housing, and all of the components located in or on the housing, the AED comprising two or more angular motion sensors that allow the AED to determine the relative orientation of the AED electrodes and to automatically select between adult doses and paediatric doses of defibrillation shocks based on the orientations of the AED electrodes.

In one aspect, the present disclosure may provide an AED, comprising
two pads to be placed on a patient,
two electrodes on the two pads respectively,
two angular motion sensors associated with the two pads respectively,
a shock generation circuit to generate doses of defibrillation shocks, and
a processor with memory and power supply, the processor configured to
   analyse angular motion signals from the two angular motion sensors to determine orientations of the two pads relative to one another,
   analyse electrocardiogram (ECG) signals from the two electrodes to determine shockable cardiac rhythms,
   automatically select between adult doses and paediatric doses of defibrillation shocks to be delivered by the two electrodes based, at least in part, on the orientations of the two pads.

According to a further aspect, the disclosure may provide an AED, comprising
two pads for placement on a patient, each pad comprising
   at least one electrode, and
   an angular motion sensor,
wherein at least one pad comprises:
a shock generation circuit configured to generate a dose of defibrillation shock, and
a processor with memory and power supply,
wherein the processor is configured to
   analyse an angular motion signal from each of the two angular motion sensors to determine an orientation of the two pads relative to one another,
   analyse electrocardiogram (ECG) signals from the two electrodes to determine shockable cardiac rhythms, and
   automatically select between an adult dose and an infant dose of defibrillation shock to be delivered by the two electrodes based, at least in part, on the relative orientation of the two pads.

In another aspect the disclosure provides a method of using an AED, comprising
receiving angular motion signals from the two angular motion sensors associated with two pads placed on a patient to deliver defibrillation shocks by two electrodes on the two pads respectively,
analysing the angular motion signals to determine orientations of the two pads relative to one another,
automatically selecting between adult doses and paediatric doses of defibrillation shocks to be delivered by the two electrodes based, at least in part, on the orientations of the two pads.

In another aspect the disclosure provides a method of using an AED, comprising
receiving an angular motion signal from each of at least two angular motion sensors, wherein each sensor is located on a pad placed on a patient, and each pad comprises an electrode,
analysing the angular motion signals to determine an orientation of the two pads relative to one another,
automatically selecting between an adult dose and an infant dose of a defibrillation shock to be delivered by the two electrodes based, at least in part, on the relative orientation of the two pads.

The following configurations may relate to any of the above aspects.

In one configuration each angular motion sensor may comprise one or more of an inertial measurement unit (IMU), a gyroscope sensor, and an accelerometer.

In one configuration the infant dose of defibrillation shocks may have lower energy than the adult dose of defibrillation shock.

In one configuration the processor may be configured to automatically select the adult dose of defibrillation shock if the two pads are determined to have an anterior-anterior orientation so that one of the two electrodes is a sternal electrode and the other is an apex electrode.

In one configuration the processor may be configured to automatically select the infant dose of defibrillation shocks if the two pads are determined to have an anterior-posterior orientation so that one of the two electrodes is a front chest electrode and the other is a back electrode.

In one configuration the process may be further configured to provide a fail-safe indication of whether the patient is an adult or an infant.

In one configuration transthoracic impedance (TTI) measurements may be acquired from the two electrodes, and the processor may be further configured to analyse the TTI measurements to provide a fail-safe indication of whether the patient is an adult or an infant.

In one configuration the processor may be further configured to automatically select between the adult dose and the paediatric dose of defibrillation shock to be delivered by the two electrodes based on a combination of the relative orientation of the two pads, and the analysis of the TTI measurements from the two electrodes.

In one configuration each pad may comprise at least three angular motion sensors, and the processor may be further configured to analyse angular motion signals from a majority of the angular motion sensors on each pad to provide a fail-safe indication of whether the patient is an adult or an infant.

In one configuration the processor may be further configured to automatically select between the adult dose and the paediatric dose of defibrillation shock to be delivered by the two electrodes based on a combination of the relative orientation of the two pads, and the analysis of the angular motion signals from the majority of the angular motion sensors on each pad.

In one configuration the method may further comprise providing a fail-safe indication of whether the patient is an adult or an infant.

In one configuration the method may further comprise
receiving transthoracic impedance (TTI) measurements from the two electrodes, and
analysing the TTI measurements from the two electrodes to provide a fail-safe indication of whether the patient is an adult or an infant.

In one configuration the method may further comprise automatically selecting between the adult dose and the infant dose of defibrillation shock to be delivered by the two electrodes based on a combination of the relative orientation of the two pads, and analysis of the TTI measurements from the two electrodes.

In one configuration the method may further comprise
receiving angular motion signals from at least three angular motions sensors located on each pad, and
analysing the angular motion signals received from a majority of the angular motion sensors on each pad to provide a fail-safe indication of whether the patient is an adult or an infant.

In one configuration the method may further comprise automatically selecting between the adult dose and the paediatric dose of defibrillation shock to be delivered by the two electrodes based on a combination of the relative orientation of the two pads, and the analysis of the angular motion signals from the majority of the angular motion sensors on each pad.

The term “axis” as used in this specification means the axis of revolution about which a line or a plane may be revolved to form a symmetrical shape. For example, a line revolved around an axis of revolution will form a surface, while a plane revolved around an axis of revolution will form a solid.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

As used herein the term “and/or” means “and” or “or”, or both.

As used herein “(s)” following a noun means the plural and/or singular forms of the noun.

For the purpose of this specification, where method steps are described in sequence, the sequence does not necessarily mean that the steps are to be chronologically ordered in that sequence, unless there is no other logical manner of interpreting the sequence.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying drawings.

### BRIEF DESCRIPTION OF DRAWINGS

Embodiments of the invention will now be described by way of example only with reference to the accompanying drawings.

FIG. 1 is a block diagram of an AED according to an embodiment of the present invention.

FIG. 2 is a schematic diagram of the AED in use on an adult.

FIGS. 3A and 3B are schematic diagrams of the AED in use on a child.

FIGS. 4A and 4B are schematic diagrams indicating the relative orientation of the pads, pre and post-snap, respectively.

FIG. 5 is a schematic diagrams depicting the orientation of the pads when the AED is used on a child.

FIG. 6 is a block diagram showing information flow when using an additional processor in the AED.

FIG. 7 is a block diagram showing information flow when using an additional processor as depicted in FIG. 6 with the addition of a hardware filter.

FIG. 8 is a flow diagram depicting process flow from snap through to shock when using the AED.

### DESCRIPTION OF EMBODIMENTS

Referring to FIG. 1, an AED 100 according to an embodiment of the present invention may generally comprise two defibrillation pads 110 to be placed on a patient. The two pads 110 may be connected by a lead 120. Two electrodes 130 may be provided on the two pads respectively. That is, each pad 110 may comprise at least one electrode 130. Two or more angular motion sensors 140 may be associated with the two pads 110 respectively. That is, each pad 110 may comprise at least one angular motion sensor 140. The two angular motion sensors 140 may comprise IMUs, gyroscope sensors, accelerometers, or a combination thereof.

An electronics module 150 may be packaged inside one or both of the two pads 110. The electronics module 150 may comprise a shock generation circuit to generate doses of defibrillation shocks, a processor with memory, and a power supply, such as a battery. Each of the shock generation circuit, the processor, and the power supply may be located in one pad 110 or distributed across the two pads 110. A suitable compact AED 100 is described in further detail in the present applicant's WO 2018/232450 which is hereby incorporated by reference in its entirety.

The processor may be configured to analyse angular motion signals from the two angular motion sensors 140 to determine orientations of the two pads 110 relative to one another. The processor may further be configured to automatically select between adult doses and paediatric doses (also referred to as infant doses, wherein the weight of the patient is within the range of about 1.5 kg to about 9.5 kg) of defibrillation shocks to be delivered by the two electrodes 130 based, at least in part, on the orientations of the two pads 110. The paediatric doses of defibrillation shocks may have lower energy than the adult doses of defibrillation shocks.

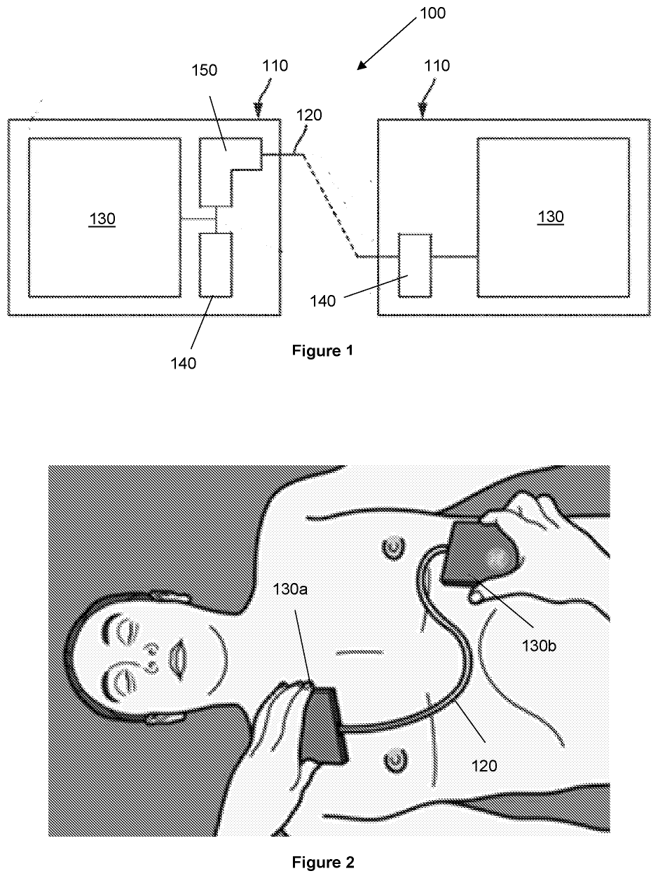

For example, as shown in FIG. 2, the processor may be configured to automatically select adult doses of defibrillation shocks if the two pads 110 are determined to have an anterior-anterior orientation so that one of the two electrodes 130 is a sternal electrode 130a and the other is an apex electrode 130b.

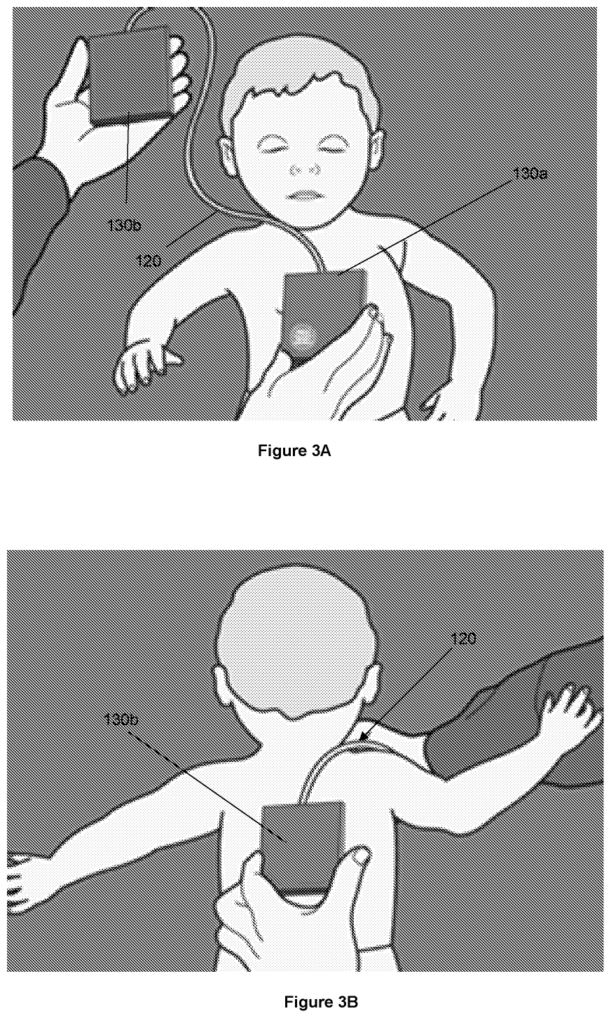

Alternatively, as shown in FIGS. 3A and 3B, the processor may be configured to automatically select paediatric doses of defibrillation shocks if the two pads 110 are determined to have an anterior-posterior orientation so that one of the two electrodes 130 is a front chest electrode 130a (FIG. 3A) and the other is a back electrode 130b (FIG. 3B).

The processor may also be configured to analyse ECG signals from the two electrodes 130 to determine shockable cardiac rhythms.

The AED 100 may be configured to provide or include a fail-safe indication of whether a patient is an adult or an infant.

If the processor establishes that the angular motion signals acquired from the at least two angular motion sensors 140 may not accurately determine the orientation of the two pads, or in cases where there is no affirmative indication of the orientation of the pads, or whether the patient is an adult or an infant, the AED may be configured to provide or include a fail-safe indication of whether a patient is an adult or an infant.

TTI measurements may also be acquired from the two electrodes, and the processor may be further configured to analyse the TTI measurements. One of the fail-safe indications provided in the AED may include, based on the TTI measurements values, determining the age group of a patient (i.e., whether the patient is an infant or an adult).

The processor may be further configured to automatically select between adult doses and paediatric doses of defibrillation shocks to be delivered by the two electrodes 130 based on a combination of the orientations of the two pads 110, and analysis of the TTI measurements from the two electrodes 130.

It is noted that the main factors of concern with respect to determining whether a patient is an adult or an infant relate to pad size, body dimensions, age and to a lesser extent, pilosity, as other factors are consistent between the two groups. Body dimensions and age are often linked in that as people age they increase in size, but also people who are larger overall will have higher TTIs than their smaller counterparts. Pilosity is the amount of hair on the chest, affects the ability for the electrode pads to fully contact the chest resulting in increased possible range for adult TTIs.

In order to determine the accurate dosage (i.e., paediatric dosage or adult dosage) of defibrillation shock, the processor may be configured to analyse both the orientation data received from the angular motion signals from the at least two angular motion sensors, and the TTI measurements to determine whether the patient is an adult or an infant.

The processor may establish that the data received from both the at least two angular motion sensors 140, and the TTI measurements are not in agreement as to whether the patient is an adult or an infant. The processor may be configured to switch the dosage to that of an adult patient. This is so that while there are potential risks associated with delivering an adult dosage to an infant, defibrillation will still be effective and achieve the desired result of arresting the arrythmia. Conversely, if an adult received an infant dosage they may not receive sufficient defibrillation energy and the shock will be ineffective.

TTI requires either a test pulse (current) to be sent between the pads or a shock to be delivered. Of notable importance to the utility of TTI as a determinant of patient age (adult vs. infant) is the effect of pad size on TTI measurement. In traditional defibrillators there are typically separate pads for paediatric (i.e., infants) and adult patients, with paediatric pads typically being smaller to account for the smaller body size. Decreasing pad size has been shown to increase TTI, with results obtained showing similar or even higher TTI for infant/paediatric compared to adults with traditional pads. This would make it near impossible for traditional defibrillators, especially the ones with different sized pads for adult and infant patients, to differentiate between dosage based on TTI as the two ranges would overlap significantly.

The present disclosure may provide a AED which uses integrated pads, and hence the pad size is held constant for all patients. As TTI is known to increase with increasing body size, the TTI for adults vs children vs infants would be expected to reflect this relationship. The claimed AED is configured to employ additional safety features in the form of a failsafe measure for its automatic dose selection that is not available in other defibrillators.

Furthermore, as the measure of TTI is so variable and related to multiple factors, it would be difficult to accurately detect the age or size of the patient with any significant granularity based on this measure alone. Consequently, in the present disclosure the measure of TTI is treated as a threshold value, above which the person is considered an adult and below, an infant.

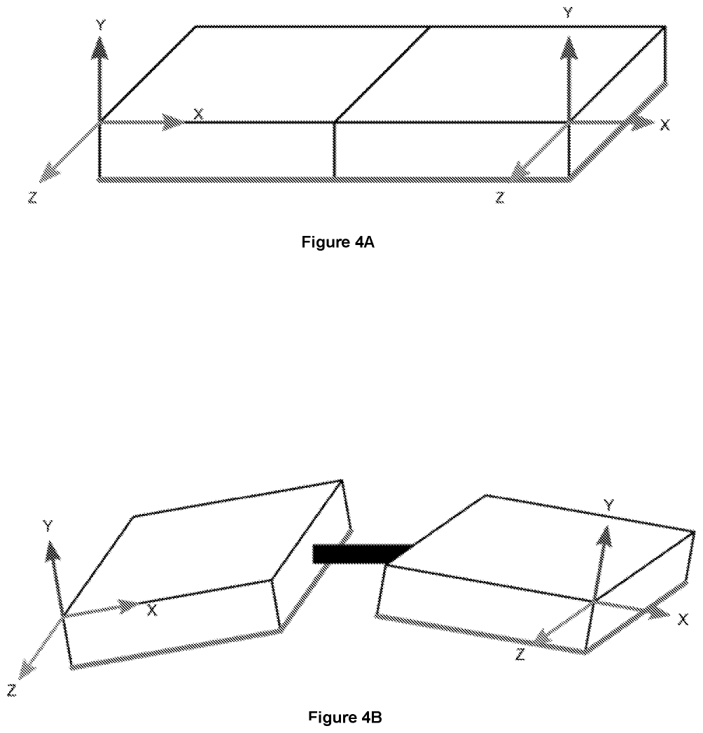

Referring to FIGS. 4A and 4B, the determination of the orientation of the pads is disclosed. The mathematics for determining orientation can, to some degree, be simplified to discrete ranges within which values will fall. The FIGS. 4A and 4B disclose an AED 100, with at least two motion sensors embedded in each of the two pads. The co-ordinate systems of each pad are labelled as x, y and z, respectively.

As will be appreciated by a skilled addressee, these coordinate systems are examples only, i.e., the sensors can feasibly be placed in any orientation within the pads provided that orientation is known. For example, in a situation, if it is thought to be necessary to put the sensors in an orientation that does not align with the AED 100 directly, a mathematical transformation is applied to address the ambiguity.

Consequently, the AED 100 may provide an indication of the relative location of the pads in space based on the orientation of the coordinate systems. The initial orientation of the pads is shown in FIG. 4A, while after snap, the orientation of the pads is shown in FIG. 4. The grey line in this figure indicates the gel on the bottom of the device. Both the pads are connected by an electrical contact (for e.g., a wire) as shown in black.

For an adult patient, the pads may be expected to broadly be in alignment in the z-direction. In both the FIGS. 4A and 4B, the y-direction as indicated by labels x, y and z, respectively. This is based on a conventional axis orientation, though in some implementations, this may be termed the z-direction. It is to be noted that while there may be some variation in the exact orientation of the axes as a result of body shape and pad position, practically, the orientation will have both z-axes aligned positively.

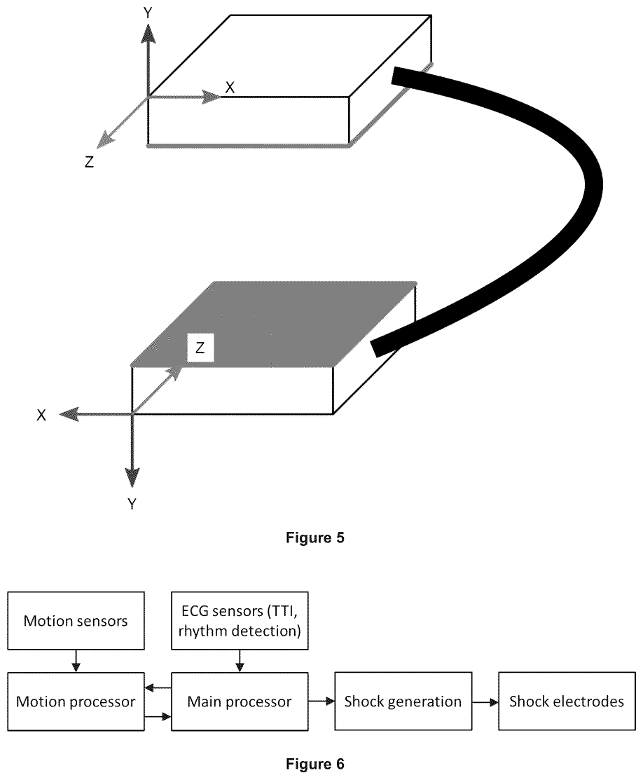

Referring to FIG. 5, for the infant patients, the pads may be positioned in an anterior-posterior directions, meaning the two pads may have opposite orientations. The relative orientation of the two pads would hence be positive/negative, while adult positioning is positive/positive. It should be appreciated that there may still be some variation in the placement of infant pads, however this would be much less susceptible to pad placement errors than on adults as there are less options regarding where to place the pads in infants.

The method for orientation determination of the sensors may vary slightly between sensors. However, in majority of cases the requirement may be to collect data and then process the collected data to get the resultant outputs. Consequently, the extent of the data needed may differ between each use-case scenario.

An accelerometer, for example, may be configured to measure the acceleration due to gravity to orient itself. As gravity always acts in the same direction, the AED 100 at rest (placed gel side down on a surface, not moving) may register an acceleration of −9.8 ms⁻² in the z-direction or −1 g. If that orientation is altered but the device left in position (e.g. placed on its side) then that acceleration would register across multiple axes to create a resultant acceleration vector of the same value. Based on the above values, and by also knowing the initial orientation of the sensors within the device, the orientation of the accelerometer vertically may be determined from the gravity vector alone, and therefore, may not strictly require the recording of data from the snap event of the AED 100.

For a gyroscope-based sensor, the orientation detection method may vary from the above. For example, in the case of gyroscope based sensors, the determination of the orientation is based on having known where the sensors are placed initially, and recording the snap event to track changes in orientation up until pad placement. As relative orientation is the main concern of this application any error introduced by the snap can be accounted for by comparing the two pads.

Referring to FIG. 6, the determination of the orientation of the pads and the requisite shock in the AED 100 is shown. The AED may further comprise a dedicated motion analysis processor configured to receive and process data received from the angular motion sensors in each pad. The dedicated angular motion analysis processor may be configured to determine orientation or motion of the pads relative to each other.

Some of the difficulties in accurately measuring the orientation of the sensors, especially for the sensor types which require recording from the snap event to placement, include additional processing power. For example, the data from such type of sensors would need to be stored for processing or processed live, which may decrease the storage requirements but increase the computational power.

Furthermore, in the case of an infant placement there may be additional steps involved in placing the pads in the posterior side (roll the body over, removal of clothes from back versus front), which may increase the time sensors need to record to be able to track the orientation/position of the pad. This in turn may increase the computational power required. Further, if the motion sensors provide a new determination after each classification cycle (in the event of multiple shocks) then this data may need to be collected continually.

The flow of information through the AED towards determining orientation and the requisite shock may take two paths. In the first path, the data may be processed using the processor (also referred to as the main processor).

In an alternate path, the data may be processed using the dedicated motion analysis processor to avoid any delay in the classification of the data received from the angular motion sensors, as this is the most important process for therapy, and is time-sensitive.

As depicted in FIG. 6, the dedicated angular motion processor receives and feeds the relevant data to the main processor (referred to as the processor) after determining the appropriate dose. The processor may also send a signal back to the motion analysis processor to prompt recalculation of orientation after pads have been adjusted. Once the processor the dosage based on the orientation of the pads, the dosage information may then be fed to the shock generation circuit to generate appropriate shock required in each of the electrodes.

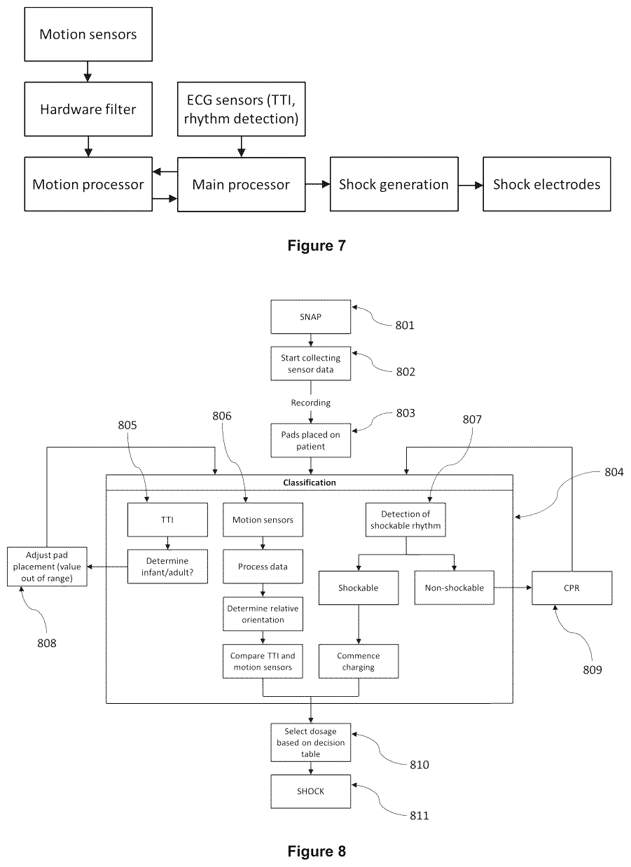

Furthermore, it will be appreciated that there is often noise present in data collected from motion sensors. The present disclosure may provide a AED configured to measure the noise from the sensors and removed said noise by applying signal processing techniques, such as digital or analog filters. In some instances, the filters may be hardware filters. In other instances, the filters may be software based. FIG. 7

FIG. 7 is an illustration in the form of a block diagram showing the information flow of FIG. 6, with an addition of a hardware filter which is configured to receive data from the motion sensors and process/eliminate the noise before passing on to the motion processor.

Referring to FIG. 8, the process flow from snap through to shock when using the AED is illustrated in the form of a flowchart. First, at step 801 a determination may be made as to whether the pads have been snapped apart from each other, in their original position. Upon the determination that the pads may be snapped, at step 802, the sensors are activated and are actively recording data. At step 803, the pads may then be placed on to a patient. Step 804 is the classification stage where the AED may determine the type of patient being treated, and the defibrillation dosage required for the patient. The classification step 804, may be divided into three simultaneous blocks, namely, (i) TTI, (ii) motion sensor, and (iii) ECG analysis.

TTI block (step 805) may measure and compares the TTI values in order to determine if the pads are placed on an adult or an infant, with additional existing checks for whether the TTI indicates incorrect placement, prompting adjustment. Motion sensor block (step 806) may process the received data from the angular motion sensors in order to determine the relative orientation of the two pads. At the next phase, the determination of patient obtained based on the orientation of the pads is compared with the determination of patient obtained based on the TTI values.

At step 807, the ECG block may be configured to determine if there is a shockable rhythm. If no shockable rhythm is detected then the device may prompt according to its usual protocols, including, but not limited to commencing CPR. On the contrary, if a shockable rhythm is detected, the shock generation circuit may commence charging the pads for a shock.

While charging the pads, at step 810 processor may select the appropriate dosage based on the Table 1. As illustrated in Table 1, the device may be configured to prioritise an adult dosage over an infant dosage in cases where the measurements from the motion sensors and the TTI are not in agreement with each other.

TABLE 1
Dosage decision table.

| Motion Sensor | TTI | Outcome |
|---|---|---|
| Adult | Adult | Adult |
| Infant | Infant | Infant |
| Adult | Infant | Adult |
| Infant | Adult | Adult |

Additionally, the classification stage also has check at step 808, wherein if no shockable rhythm is detected and the TTI or motion sensors indicate poor placement the user may be directed to adjust the pad placement. Further check is also performed at step 809, wherein if it is determined during the classification stage that no shockable rhythm is detected, then this information may be relayed, such that the user may perform alternate therapeutic measures such as CPR, etc.

In the use-case scenarios where the determined orientation of the sensors may indicate out of range values, the device may be configured to default to an adult shock.

The AED 100 may further be configured to provide a fail-safe indication of whether a patient is an adult or an infant based on data received from
i) a potentiometer, or
ii) an inductor, or
iii) a plurality of line of sight sensors, or
iv) by the placement of a physical cable between the two pads, or
v) any combination of two or more of (i) to (iv), and assessing the distance between the two pads.

The AED 100 may further comprise at least three angular motion sensors placed in each of the pads. In these implementations, the AED may be configured to provide a fail-safe indication of whether a patient is an adult or an infant based on the data received from each sensor to enable redundancy in case of sensor failure or calibration issues.

In the above implementation where at least three angular motion sensors are placed in each of the pads, the results from all the three sensors would be calculated separately and a majority rule (i.e. two out of three sensors indicate an agreement in the orientation) may be used to select the dosage. It may be preferable in this scenario that at least one sensor is an IMU as these can combine output from an accelerometer and gyroscope for greater accuracy. Furthermore, in this implementation, due to the increased number of sensors it may be possible to increase the threshold for infant TTI values to enable greater accuracy in selecting dosage.

Furthermore, in implementations where multiple sensors are used, it may be possible that multiple sensors may malfunction or return incorrect results. This could be due to a number of reasons, however, under a majority rule system two faulty sensors on one pad could result in a number of different scenarios. In most of the cases, the pad positioning would become indeterminate which would result in an adult dose as that is the best therapy selection.

In an alternate scenario where two faulty sensors may override the functioning sensor and return the opposite orientation of the pads
on an infant this may return an adult dosage determination which is of low risk, and
on an adult this may result in the motion sensors returning an infant determination (in this situation, if the TTI also returns an infant value this could lead to an infant dosage being administered to an adult which may not be effective).

TTI tends to increase under most factors that differentiate adults from infants and as such it is unlikely that TTI would also fail to return an accurate reading.

An additional scenario may be defibrillation on a small child, who may have a sufficiently low TTI to register as infant. In this case, an infant dose may be administered to a child, however this is of low risk as many defibrillators deliver low dose shocks to children, not just infants. Due to this however, TTI would be unable to be removed as a failsafe measure.

Therefore, in the present device, the use of multiple sensors would create a more accurate reading and may lead to being able to increase the TTI threshold due to greater confidence in the motion sensor result.

In the present disclosure, as the sensors may be configured to measure the relative orientation of the pads they can be placed in any known, fixed position and orientation within the pads. That said, however, it may be preferable to mount the sensors on the PCB itself as it may provide the most stable attachment point for the sensors.

The AED may comprise one sensor placed on the controller side of the board (with the battery and processors) and the other on a capacitor bank side. The device may also comprise an additional wire added to the cable connecting the two pads to transmit the data to the controller side.

Each pad of the present defibrillator may have a volume of about 100, 110, 120, 130, 140, 150, 160, 170, 180, 190 or 200 cm³, and useful ranges may be selected from any of these values (for example, about 100 to about 200, about 100 to about 180, about 100 to about 160, about 100 to about 150, about 110 to about 200, 110 to about 190, about 110 to about 170, about 110 to about 150, about 120 to about 200, about 120 to about 180, about 120 to about 150, about 130 to about 200, about 130 to about 180, about 130 to about 150, about 140 to about 200, about 140 to about 180, about 140 to about 160 or about 140 to about 150 cm³).

Each pad of the present defibrillator may have a surface area of about 50, 60, 70, 80, 90 or 100 cm², and useful ranges may be selected from any of these values (for example, about 50 to about 100, about 50 to about 80, about 50 to about 70, about 50 to about 60, about 60 to about 100, about 60 to about 80, about 60 to about 70 or about 50 to about 60 cm²).

Described is a compact AED configured for a single use defibrillation therapy and resuscitation of a patient. The device is configured to automatically select between an adult dose and an infant dose of defibrillation shock to be delivered by the two electrodes based, at least in part, on the relative orientation of the two pads. The resultant device is user friendly, requires minimal training or knowledge in terms of its operational use. Thus, removing points of confusion for the user unfamiliar with the device.

For example, the volume of each of the pads may be about 9.7 cm×9.3 cm×1.7 cm to give a total volume of 153 cm³, and the surface area may be about 8.2 cm×8.6 cm to give a total surface area of 70.5 cm²

Embodiments of the present invention provide AEDs that are both generally and specifically useful for automatically selecting between adult and paediatric doses of defibrillation shocks.

Embodiments of the present invention advantageously omit the space-consuming, manual fail-safe arrangements used in conventional AEDs to manually select between adult and paediatric doses of defibrillation shocks. The omission of manual fail-safe arrangements may advantageously increase (or “buy back”) internal packaging space available for batteries, which in turn may increase the cycle life and shelf life of compact AEDs.

In other words, the automatic selection between adult and paediatric doses may facilitate optimal use of internal packaging space in compact AEDs for power supply. For example, embodiments of the present invention may facilitate compact AEDs having a target shelf life of up to at least one or two years.

Although embodiments have been described with reference to a number of illustrative embodiments thereof, it will be understood by those skilled in the art that various changes in form and details may be made therein without departing from the spirit and scope of the invention as defined by the appended claims.

Many modifications will be apparent to those skilled in the art without departing from the scope of the present invention as herein described with reference to the accompanying drawings.

1. An automated external defibrillator (AED), comprising two pads for placement on a patient, each pad comprising:
at least one electrode, and
an angular motion sensor,
wherein at least one pad comprises:
a shock generation circuit configured to generate a dose of defibrillation shock, and
a processor with memory and power supply, wherein the processor is configured to:
analyse an angular motion signal from each of the two angular motion sensors to determine an orientation of the two pads relative to one another,
analyse electrocardiogram (ECG) signals from the two electrodes to determine shockable cardiac rhythms, and
automatically select between an adult dose and an infant dose of defibrillation shock to be delivered by the two electrodes based, at least in part, on the relative orientation of the two pads.

2. The AED of claim 1, wherein each angular motion sensor comprises one or more of an inertial measurement unit (IMU), a gyroscope sensor, and an accelerometer.

3. The AED of claim 1, wherein the infant dose of defibrillation shock has lower energy than the adult dose of defibrillation shock.

4. The AED of claim 1, wherein the processor is configured to automatically select the adult dose of defibrillation shock if the two pads are determined to have an anterior-anterior orientation so that one of the two electrodes is a sternal electrode and the other is an apex electrode.

5. The AED of claim 1, wherein the processor is configured to automatically select the infant dose of defibrillation shock if the two pads are determined to have an anterior-posterior orientation so that one of the two electrodes is a front chest electrode and the other is a back electrode.

6. The AED of claim 1, wherein the processor is further configured to provide a fail-safe indication of whether the patient is an adult or an infant.

7. The AED of claim 1, wherein transthoracic impedance (TTI) measurements are acquired from the two electrodes, and the processor is further configured to analyse the TTI measurements to provide a fail-safe indication of whether the patient is an adult or an infant.

8. The AED of claim 7, wherein the processor is further configured to automatically select between the adult dose and the infant dose of defibrillation shock to be delivered by the two electrodes based on a combination of the relative orientation of the two pads, and the analysis of the TTI measurements from the two electrodes.

9. The AED of claim 1, wherein each pad comprises at least three angular motion sensors, and wherein the processor is further configured to analyse angular motion signals from a majority of the angular motion sensors on each pad to provide a fail-safe indication of whether the patient is an adult or an infant.

10. The AED of claim 9, wherein the processor is further configured to automatically select between the adult dose and the infant dose of defibrillation shock to be delivered by the two electrodes based on a combination of the relative orientation of the two pads, and the analysis of the angular motion signals from the majority of the angular motion sensors on each pad.

11. A method of using an automated external defibrillator, comprising
receiving an angular motion signal from each of at least two angular motion sensors, wherein each sensor is located on a pad placed on a patient, and each pad comprises an electrode,
analysing the angular motion signals to determine an orientation of the two pads relative to one another,
automatically selecting between an adult dose and an infant dose of a defibrillation shock to be delivered by the two electrodes based, at least in part, on the relative orientation of the two pads.

12. The method of claim 11, wherein each angular motion sensor comprises one or more of an inertial measurement unit (IMU), a gyroscope sensor, and an accelerometer.

13. The method of claim 11, wherein the infant dose of defibrillation shock has lower energy than the adult dose of defibrillation shock.

14. The method of claim 11, further comprising automatically selecting the adult dose of defibrillation shock if the two pads are determined to have an anterior-anterior orientation so that one of the two electrodes is a sternal electrode and the other is an apex electrode.

15. The method of claim 11, further comprising automatically selecting an infant dose of defibrillation shock if the two pads are determined to have an anterior-posterior orientation so that one of the two electrodes is a front chest electrode and the other is a back electrode.

16. The method of claim 11, further comprising providing a fail-safe indication of whether the patient is an adult or an infant.

17. The method of claim 11, further comprising:
receiving transthoracic impedance (TTI) measurements from the two electrodes, and
analysing the TTI measurements from the two electrodes to provide a fail-safe indication of whether the patient is an adult or an infant.

18. The method of claim 17, further comprising automatically selecting between the adult dose and the infant dose of defibrillation shock to be delivered by the two electrodes based on a combination of the relative orientation of the two pads, and the analysis of the TTI measurements from the two electrodes.

19. The method of claim 11, further comprising:
receiving angular motion signals from at least three angular motions sensors located on each pad, and
analysing the angular motion signals received from a majority of the angular motion sensors on each pad to provide a fail-safe indication of whether the patient is an adult or an infant.

20. The method of claim 19, further comprising automatically selecting between the adult dose and the infant dose of defibrillation shock to be delivered by the two electrodes based on a combination of the relative orientation of the two pads, and the analysis of the angular motion signals from the majority of the angular motion sensors on each pad.
